(19) United States
(12) Patent Application Publication
(10) Pub. No.: US 2004/0052785 A1
(43) Pub. Date: Mar. 18, 2004

(54) Combination therapy using receptor tyrosine kinase inhibitors and angiogenesis inhibitors

(76) Inventors: Simon Goodman, Griesheim (DE); Hans-Georg Kreysch, Mainz (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/250,783

(22) PCT Filed: Dec. 21, 2001
(86) PCT No.: PCT/EP01/15241

(30) Foreign Application Priority Data
Jan. 9, 2001 (EP) 01100507.1

(51) Int. Cl. A61K 39/395

(52) U.S. Cl. 424/143.1; 424/146.1

(57) ABSTRACT

The invention relates to a combination therapy for the treatment of tumors and tumor metastases comprising administration of receptor tyrosine kinase antagonists/inhibitors, especially ErbB receptor antagonists, more preferably EGF receptor (Her 1) antagonists and anti-angiogenic agents, preferably integrin antagonists, optionally together with agents or therapy forms that have additive or synergistic efficacy when administered together with said combination of antagonists/inhibitors, such as chemotherapeutic agents and or radiation therapy. The therapy can result in a synergistic potential increase of the inhibition effect of each individual therapeutic on tumor cell proliferation, yielding more effective treatment than found by administering an individual component alone.

### TECHNICAL FIELD OF THE INVENTION

The invention relates to a combination therapy for the treatment of tumors and tumor metastases comprising administration of receptor tyrosine kinase antagonists/inhibitors, especially ErbB receptor antagonists, more preferably EGF receptor (Her 1) antagonists and anti-angiogenic agents, preferably integrin antagonists, optionally together with agents or therapy forms that have additive or synergistic efficacy when administered together with said combination of antagonists/inhibitors, such as chemotherapeutic agents and or radiation therapy. The therapy can result in a synergistic potential increase of the inhibition effect of each individual therapeutic on tumor cell proliferation, yielding more effective treatment than found by administering an individual component alone.

### BACKGROUND OF THE INVENTION

The epidermal growth factor receptor (EGF receptor or EGFR), also known as c-erbB1/Her 1, and the product of the neu oncogene (also known as c-erbB2/Her 2) are the members of the EFG receptor super family, which belongs to the large family of receptor tyrosine kinases. They interact at the cell surface with specific growth factors or natural ligands, such as EGF or TGF alpha, thus, activating the receptor tyrosine kinase. A cascade of downstream signaling proteins are activated in general leading to altered gene expression and increased growth rates.

C-erbB2 (Her 2) is a transmembrane tyrosine kinase having a molecular weight of about 185.000, with considerable homology to the EGF receptor (Her 1), although a specific ligand for Her 2 has not yet been clearly identified so far.

The EGF receptor is a transmembrane glycoprotein which has a molecular weight of 170.000, and is found on many epithelial cell types. It is activated by at least three ligands, EGF, TGF-α (transforming growth factor alpha) and amphiregulin. Both epidermal growth factor (EGF) and transforming growth factor-alpha (TGF-a) have been demonstrated to bind to EGF receptor and to lead to cellular proliferation and tumor growth. These growth factors do not bind to Her 2 (Ulrich and Schlesinger, 1990, Cell 61, 203). In contrast to several families of growth factors, which induce receptor dimerization by virtue of their dimeric nature (e.g. PDGF) monomeric growth factors, such as EGF, contain two binding sites for their receptors and, therefore, can cross-link two neighboring EGF receptors (Lemmon et al., 1997, EMBO J. 16, 281). Receptor dimerization is essential for stimulating of the intrinsic catalytic activity and for the autophosphorylation of growth factor receptors. It should be remarked that receptor protein tyrosine kinases (PTKs) are able to undergo both homo- and heterodimerization.

Clinical studies indicate that both EGF receptor and c-erbB2 are overexpressed in certain types of tumors, especially, breast, ovary, bladder, colon, kidney, head and neck cancers and squamous carcinomas of the lung. (Mendelsohn, 1989, Cancer Cells 7, 359; Mendelsohn, 1990, Cancer Biology 1, 339). Therefore, these observations have stimulated preclinical investigations targeting on inhibiting the function of human EGF receptors or c-erbB2 as novel therapeutic approaches to treat cancer (see e.g. Baselga et al., 1996, J. Clin. Oncol. 14, 737; Fan and Mendelsohn, 1998; Curr. Opin. Oncol. 10, 67). It has been reported that, for example, anti-EGF receptor antibodies as well as anti-Her 2 antibodies show fruitful results in human cancer therapy. Thus, humanized monoclonal antibody 4D5 (hMAb 4D5, HERCEPTIN®) is already a commercialized product.

It has been demonstrated that anti-EGF receptor antibodies while blocking EGF and TGF-a binding to the receptor appear to inhibit tumor cell proliferation. In view of these findings, a number of murine and rat monoclonal antibodies against EGF receptor have been developed and tested for their ability inhibit the growth of tumor cells in vitro and in vivo (Modjtahedi and Dean, 1994, J. Oncology 4, 277). Humanized monoclonal antibody 425 (hMAb 425) (U.S. Pat. No. 5,558,864; EP. 0531 472) and chimeric monoclonal antibody 225 (cMAb 225) (Naramura et al., 1993, Cancer Immunol. Immunother. 37, 343-349, WO 96/40210), both directed to the EGF receptor, have shown their efficacy in clinical trials. The C225 antibody was demonstrated to inhibit EGF-mediated tumor cell growth in vitro and inhibit human tumor formation in vivo in nude mice. The antibody, moreover, appeared to act, above all, in synergy with certain chemotherapeutic agents (i.e., doxorubicin, adriamycin, taxol, and cisplatin) to eradicate human tumors in vivo in xenograft mouse models. Ye et al. (1999, Oncogene 18, 731) have reported that human ovarian cancer cells can be treated successfully with a combination of both cMAb 225 and hMAb 4D5.

Angiogenesis, also referred to as neovascularization, is a process of tissue vascularization that involves the growth of new blood vessels into a tissue. The process is mediated by the infiltration of endothelial cells and smooth muscle cells. The process is believed to proceed in any one of three ways: (1) the vessels can sprout from pre-existing vessels; (2) de novo development of vessels can arise from precursor cells (vasculogenesis); or (3) existing small vessels can enlarge in diameter (Blood et al., 1990, Bioch. Biophys. Acta 1032, 89. Vascular endothelial cells are known to contain at least five RGD-dependent integrins, including the vitronectin receptor (αᵥβ₃ or αᵥβ₅), the collagen Types I and IV receptor, the laminin receptor, the fibronectin/laminin/collagen receptor and the fibronectin receptor (Davis et al., 1993, J. Cell. Biochem. 51, 206). The smooth muscle cell is known to contain at least is six RGD-dependent integrins, including αᵥβ₃αᵥβ₅.

Inhibition of cell adhesion in vitro using monoclonal antibodies immunospecific for various integrin α or β subunits have implicated the vitronectin receptor αᵥβ₃ in cell adhesion of a variety of cell types including microvascular endothelial cells (Davis et al., 1993, J. Cell. Biol. 51, 206).

Integrins are a class of cellular receptors known to bind extracellular matrix proteins, and mediate cell-extracellular matrix and cell-cell interactions, referred generally to as cell adhesion events. The integrin receptors constitute a family of proteins with shared structural characteristics of non-covalent heterodimeric glycoprotein complexes formed of α and β subunits. The vitronectin receptor, named for its original characteristic of preferential binding to vitronectin, is now known to refer to three different integrins, designated αᵥβ₁, αᵥβ₃ and αᵥβ₅. αᵥβ₁ binds fibronectin and vitronectin. αᵥβ₃ binds a large variety of ligands, including fibrin, fibrinogen, laminin, thrombospondin, vitronectin and von Willebrand's factor. αᵥβ₅ binds vitronectin. It is clear that there are different integrins with different biological functions as well as different integrins and subunits having shared biological specificity and function. One important recognition site in a ligand for many integrins is the arginine-glycine-aspartic acid (RGD) tripeptide sequence. RGD is found in all of the ligands identified above for the vitronectin receptor integrins. This RGD recognition site can be mimicked by linear and cyclic (poly)peptides that contain the RGD sequence. Such RGD peptides are known to be inhibitors or antagonists, respectively, of integrin function. It is important to note, however, that depending upon the sequence and structure of the RGD peptide, the specificity of the inhibition can be altered to target specific integrins. Various RGD polypeptides of varying integrin specificity have been described, for example, by Cheresh, et al., 1989, Cell 58, 945, Aumailley et al., 1991, FEBS Letts. 291, 50, and in numerous patent applications and patens (e.g. U.S. Pat. Nos. 4,517,686, 4,578,079, 4,589,881, 4,614,517, 4,661,111, 4,792,525; EP 0770 622).

The generation of new blood vessels, or angiogenesis, plays a key role in the growth of malignant disease and has generated much interest in developing agents that inhibit angiogenesis (see, for example, Holmgren et al., 1995, Nature Medicine 1, 149; Folkman, 1995, Nature Medicine 1, 27; O'Reilly et. al., 1994, Cell 79, 315). The use of αᵥβ₃ integrin antagonists to inhibit angiogenesis is known in methods to inhibit solid tumor growth by reduction of the blood supply to the solid tumor (see, for example, U.S. Pat. No. 5,753,230 and U.S. Pat. No. 5,766,591, which describe the use of αᵥβ₃ antagonists such as synthetic polypeptides, monoclonal antibodies and mimetics of αᵥβ₃ that bind to the αᵥβ₃ receptor and inhibit angiogenesis). Methods and compositions for inhibiting αᵥβ₅ mediated angiogenesis of tissues using antagonists of the vitronectin receptor αᵥβ₅ are disclosed in WO 97/45447. Angiogenesis is characterized by invasion, migration and proliferation of endothelial cells, processes that depend on cell interactions with extracellular matrix components. In this context, the integrin cell-matrix receptors mediate cell spreading and migration. The endothelial adhesion receptors of integrin αᵥβ₃ were shown to be key players by providing a vasculature-specific target for anti-angiogenic treatment strategies (Brooks et al., 1994, Science 264, 569; Friedlander et. al., 1995, Science 270). The requirement for vascular integrin αᵥβ₃ in angiogenesis was demonstrated by several in vivo models where the generation of new blood vessels by transplanted human tumors was entirely inhibited either by systemic administration of peptide antagonists of integrin αᵥβ₃ and αᵥβ₅, as indicated above, or, alternatively, by anti-αᵥβ₃ antibody LM609 (Brooks et al., 1994, Cell 79, 1157; ATCC HB 9537). This antibody blocks the αᵥβ₃ integrin receptor the activation of which by its natural ligands promotes apoptosis of the proliferative angiogenic vascular cells and thereby disrupts the maturation of newly forming blood vessels, an event essential for the proliferation of tumors. Nevertheless, it was recently reported, that melanoma cells could form web-like patterns of blood vessels even in the absence of endothelial cells (1999, Science 285, 14), implying that tumors might be able to circumvent some anti-angiogenic drugs which are only effective in the presence of endothelial tissue.

Numerous molecules stimulate endothelial proliferation, migration and assembly, including VEGF, Ang1 and bFGF, and are vital survival factors. VEGF (Vascular Endothelial Growth Factor) has been identified as a selective angiogenic growth factor that can stimulate endothelial cell mitogenesis. VEGF, in particular, is thought to be a major mediator of angiogenesis in a primary tumor and in ischemic ocular is diseases. VEGF is a homodimer (MW: 46.000) that is an endothelial cell-specific angiogenic (Ferrara et al., 1992, Endocrin. Rev., 13, 18) and vasopermeability factor (Senger et al., 1986, Cancer Res., 465629) that binds to high-affinity membrane-bound receptors with tyrosine kinase activity (Jakeman et al., 1992, J. Clin. Invest., 89, 244). Human tumor biopsies exhibit enhanced expression of VEGF mRNAs by malignant cells and VEGF receptor mRNAs in adjacent endothelial cells. VEGF expression appears to be greatest in regions of tumors adjacent to vascular areas of necrosis. (for review see Thomas et al., 1996, J. Biol. Chem. 271(2), 603; Folkman, 1995, Nature Medicine 1, 27). WO 97/45447 has implicated the αᵥβ₅ integrin in neovascularization, particularly, that induced by VEGF, EGF and TGF-α, and discloses that αᵥβ₅ antagonist can inhibit VEGF promoted angiogenesis. Effective anti-tumor therapies may also utilize targeting VEGF receptor for inhibition of angiogenesis using monoclonal antibodies. (Witte et al.,1998, Cancer Metastasis Rev. 17(2), 155). MAb DC-101 is known to inhibit angiogenesis of tumor cells.

As summarized above it is evident that EGF, VEGF and integrins αᵥβ₃ and αᵥβ₅ and their receptors are basically involved in tumor proliferation and tumor angiogenesis, and that effective inhibitors, especially monoclonal antibodies, directed to EGF receptor and/or VEGF receptor and/or integrin receptors or any other protein tyrosine kinase receptors are principally suitable candidates for tumor therapy. Monoclonal antibodies which can specifically recognize their antigen epitopes on the relevant receptors, are of special interest.

However, the use of such antibodies, which were successful in vitro and in animal models, have not shown satisfying efficacy in patients as mono-drug therapy. Similar results were obtained when other anti-angiogenic or EGF receptor antagonists than antibodies were used in clinical trials. It seems, that tumors, if some specific sites are blocked, may use other cell surface molecules to compensate for said original blocking. Thus, tumors do not really shrink during various anti-angiogenic or anti-proliferative therapies. For these reasons, combination therapies were proposed to circumvent this problem using monoclonal antibodies together with cytotoxic or chemotherapeutic agents or in combination with radiotherapy. Indeed, clinical trials have shown that these combination therapies are more efficient than the corresponding mono-administrations. Thus, for example, antibody-cytokine fusion protein therapies have been described which promote immune response-mediated inhibition of established tumors such as carcinoma metastases. For example, the cytokine interleukin 2 (IL-2) has been fused to specific monoclonal antibodies KS1/4 and ch14.18 directed to the tumor associated antigens epithelial cell adhesion molecule (Ep-CAM, KSA, KS1/4 antigen) or the disialoganglioside GD, respectively, to form the fusion proteins ch14.18-IL-2 and KS1/4-IL-2, respectively (U.S. Pat. No. 5,650,150). Another clinical approach is based on the administration of monclonal antibody c225 in combination with Herceptin® (Ye et al, 1999, I.c.). Furthermore, the combination of anti-EGF receptor antibodies together with anti-neoplastic agents, such as cisplatin or doxorubicin, was disclosed in EP 0667 165 (A1) and U.S. Pat. No. 6,217,866); a similar combination, especially a combination of Herceptin® with cisplatin and other cytotoxic factors, was described in Genentech's U.S. Pat. No. 5,770,195. Synergy effects between an anti-angiogenic integrin a, antagonist and above-mentioned antibody-cytokine fusion proteins were observed in tumor metastases (Lode et al., 1999, Proc. Natl. Acad. Sci. 96, 1591, WO 00/47228). Methods of using integrin antagonists together with anti-neoplastic agents were recently claimed in WO 00/38665. Recently, it was found that a combination of gemcitabine with specific monoclonal antibody DC-101, which inhibits angiogenesis, increased the anti-tumor effect in pancreatic cancer of mice compared with gemcitabine alone. DE 198 42415 discloses the combination of a specific cyclic RGD peptide as integrin inhibitor with specific anti-angiogenesis agents. Other approaches suggest the administration of EGF receptor blocking agents, antibodies included, or integrin antagonists combined with radiation or radiotherapy, respectively (e.g. WO 99/60023, WO 00/0038715).

Nevertheless, although various combinations therapies are under investigation and in clinical trials, the outcome of these therapies are not fruitful enough. Therefore, it is a need to develop further combinations which can show increased efficacy and reduced side-effects.

### SUMMARY OF THE INVENTION

The present inventions describes for the first time a novel pharmaceutical treatment which is based on the new concept in tumor therapy to administer to an individual in a therapeutically effective amount an agent that blocks or inhibits a receptor tyrosine kinase, preferably an ErbB receptor and more preferably an EGF receptor together with an anti-angiogenic agent. Optionally the composition according to this invention may comprise further therapeutically active compounds, preferably selected from the group consisting of cytotoxic agents, chemotherapeutic agents and other pharmacologically active compounds which may enhance the efficacy of said agents or reduce the side effects of said agents.

Thus, the invention relates to pharmaceutical compositions comprising as preferred ErbB receptor antagonists an anti-EGFR (ErbB1/Her 1) antibody and as anti-angiogenic agent an inhibitor or antagonist of any of the aᵥβ₃, aᵥβ₅ or aᵥβ₆ integrin receptors, preferably an RGD containing linear or cyclic peptide. Especially, the inventions relates, as a preferred embodiment, to a specific combination therapy comprising anti-EGFR or anti-Her2 antibodies, such as humanized monoclonal antibody 425 (h425, EMD 72000), chimeric monoclonal antibody 225 (c225) or Herceptin® together with preferably RGD-containing integrin inhibitors, most preferably with the cyclic peptide cyclo-(Arg-Gly-Asp-DPhe-NMe-Val), optionally together with a chemotherapeutic compound.

According to this invention said therapeutically active agents may also be provided by means of a pharmaceutical kit comprising a package comprising one or more receptor tyrosine kinase antagonists, one or more anti-angiogenic agents, and optionally, one or more cytotoxic/chemotherapeutic agents in single packages or in separate containers. The therapy with this combinations may include optionally treatment with radiation.

However, the invention relates, furthermore, to a combination therapy comprising the administration of only one (fusion) molecule, having anti-receptor tyrosine kinase, preferably anti-ErbB receptor activity and anti-angiogenic activity, optionally together with one or more cytotoxic/chemotherapeutic agents. An example is an anti-EGFR is antibody, such as h425 or c225 as described above and below, which is fused at the C-terminal of its Fc portion to an anti-hormonal agent by known recombinant or chemical methods. A further example is a bispecific antibody, wherein one specificity is directed to an nuclear hormone receptor and the other one is directed to the EGF receptor.

Principally, the administration can be accompanied by radiation therapy, wherein radiation treatment can be done substantially concurrently or before or after the drug administration. The administration of the different agents of the combination therapy according to the invention can also be achieved substantially concurrently or sequentially. Tumors, bearing receptors on their cell surfaces involved in the development of the blood vessels of the tumor, may be successfully treated by the combination therapy of this invention.

It is known that tumors elicit alternative routes for their development and growth. If one route is blocked they often have the capability to switch to another route by expressing and using other receptors and signaling pathways. Therefore, the pharmaceutical combinations of the present invention may block several of such possible development strategies of the tumor and provide consequently various benefits. The combinations according to the present invention are useful in treating and preventing tumors, tumor-like and neoplasia disorders and tumor metastases, which develop and grow by activation of their relevant hormone receptors which are present on the surface of the tumor cells. Preferably, the different combined agents of the present invention are administered in combination at a low dose, that is, at a dose lower than has been conventionally used in clinical situations. A benefit of lowering the dose of the compounds, compositions, agents and therapies of the present invention administered to an individual includes a decrease in the incidence of adverse effects associated with higher dosages. For example, by the lowering the dosage of an agent described above and below, a reduction in the frequency and the severity of nausea and vomiting will result when compared to that observed at higher dosages. By lowering the incidence of adverse effects, an improvement in the quality of life of a cancer patient is contemplated. Further benefits of lowering the incidence of adverse effects include an improvement in patient compliance, a reduction in the number of hospitalizations needed for the treatment of adverse effects, and a reduction in the administration of analgesic agents needed to treat pain associated with the adverse effects. Alternatively, the methods and combination of the present invention can also maximize the therapeutic effect at higher doses.

Tumors, bearing (over-expressed) ErbB receptors, preferably ErbB1 (Her1, EGFR) or ErbB2 (Her 2) receptors on their cell surfaces, may be successfully treated by the combinations according to the inventions. The combinations within the pharmaceutical treatment according to the inventions show an astonishing synergetic effect. In administering the combination of drugs real tumor shrinking and disintegration could be observed during clinical studies while no significant adverse drug reactions were detectable. Above all, the three-drug combinations (receptor tyrosine kinase, preferably ErbB receptor blocking agent plus anti-angiogenic agent plus chemotherapeutic agent) show superior efficacy. However, whether a chemotherapeutic drug is synergistically effective or not depends on the drug itself, the receptor tyrosine kinase, preferably ErbB receptor antagonist and the tumor cell that is treated with said agents, and must be usually checked case by case.

In detail the invention refers to:

a pharmaceutical composition comprising an agent or agents having

(i) at least one receptor tyrosine kinase blocking/inhibiting specificity and

(ii) at least one angiogenesis blocking/inhibiting specificity, wherein said agent or agents is/are not a cytokine immunoconjugate, optionally together with a pharmaceutically acceptable carrier, diluent or recipient;

as a first alternative, a pharmaceutical comprising

(i) at least one agent having a receptor tyrosine kinase blocking specificity, and

(ii) at least one agent having an angiogenesis inhibiting specificity;

as a second alternative, a pharmaceutical composition, comprising an agent having a receptor tyrosine kinase blocking specificity as well as an angiogenesis inhibiting specificity.

corresponding compositions further comprising at least one cytotoxic, preferably chemotherapeutic agent;

in more detail, a pharmaceutical composition, wherein said agent (i) has a ErbB receptor blocking/inhibiting specificity;

a corresponding pharmaceutical composition, wherein the ErbB receptor specificity of said agent is related to the EGF receptor (ErbB1/Her1) or the ErbB2/Her2 receptor;

in more detail, a pharmaceutical composition, wherein said agent is an antibody or a functionally intact derivative thereof, comprising a binding site which binds to an epitope of the ErbB1 (Her1) or Erb2 (Her2) receptor;

as preferred embodiment, a pharmaceutical composition, wherein said antibody or functionally intact derivative thereof is selected from the group:

humanized monoclonal antibody 425 (h425)

chimeric monoclonal antibody 225 (c225)

humanized monoclonal antibody Her 2, the corresponding humanized, chimeric or de-immunized functionally intact dervatives included;

a corresponding pharmaceutical composition, wherein said angiogenesis inhibiting agent is an aᵥβ₃, aᵥβ₅ or an aᵥβ₆ integrin inhibitor;

a corresponding pharmaceutical composition, wherein said integrin inhibitor is an RGD-containing linear or cyclic peptide, preferably cyclo(Arg-Gly-Asp-DPhe-NMeVal);

as a specific embodiment, a pharmaceutical composition, wherein said antibody or functionally intact derivative thereof is humanized monoclonal antibody 425 (h425) or chimeric monoclonal antibody 225 (c225), de-immunized forms included, and said integrin inhibitor is cyclo(Arg-Gly-Asp-DPhe-NMeVal), optionally comprising, optionally in separate containers or packages, a chemotherapeutic agent which is selected from any of the compounds of the group: cisplatin, doxorubicin, gemcitabine, docetaxel, paclitaxel, bleomycin;

a corresponding pharmaceutical composition, wherein said integrin inhibitor is an antibody or a functionally intact derivative thereof, comprising a binding site which binds to an epitope of an integrin receptor, preferably selected from the group of antibodies: LM609, P1F6, 17E6, 14D9.F8, humanized, chimeric and de-immunized versions thereof included;

a pharmaceutical composition, wherein one of said agents is a bispecific antibody or a heteroantibody molecule comprising a first binding site that binds to an epitope of a receptor tyrosine kinase, preferably ErbB receptor, and a second binding site that binds to an epitope of an angiogenesis receptor, preferably an integrin receptor;

a specific corresponding pharmaceutical composition, wherein said monoclonal antibodies are selected from h425, c225 or Her 2, and from the monoclonal antibodies LM609, P1F6, 17E6 and 14D9.F8;

a pharmaceutical composition, wherein one of said agents is an immunoconjugate consisting of an antibody or antibody fragment, bearing one of said blocking specificities, and a non-immunological molecule, fused to the antibody or antibody fragment bearing the other specificity;

a corresponding pharmaceutical composition, wherein the antibody portion or fragment thereof comprises a binding site that binds to an epitope of an ErbB receptor, preferably an EGF receptor (Her 1), and the fused non-immunological molecule comprises a binding site that binds to an epitope of an integrin receptor;

a specific pharmaceutical composition thereof, wherein said antibody portion which binds to an epitope of an ErbB receptor is selected from monoclonal antibodies h425, c225 or Her 2, and said non-imunological portion which binds to an epitope of an integrin receptor is cyclo(Arg-Gly-Asp-DPhe-NMeVal);

a pharmaceutical kit comprising

(i) a package comprising at least one receptor tyrosine kinase inhibiting, preferably an ErbB receptor blocking agent, and

(ii) a package comprising at least one angiogenesis inhibiting agent, preferably an aᵥβ₃, aᵥβ₅ or an aᵥβ₆ integrin receptor inhibiting agent, more preferably an RGD-containing linear or cyclic peptide, especially cyclo(Arg-Gly-Asp-DPhe-NMeVal);

optionally further comprising a package comprising a cytotoxic agent;

a corresponding pharmaceutical kit, wherein said ErbB receptor blocking agent is an antibody or a functionally intact derivative thereof, having a binding site that binds to an epitope of said receptor; said antibody is preferably selected from the group of antibodies: humanized monoclonal antibody 425 (h425), chimeric monoclonal antibody 225 (c225) or humanized monoclonal antibody Her 2;

a pharmaceutical kit, wherein said angiogenesis inhibiting agent is an antibody or an active derivative thereof, preferably selected from the group of antibodies: LM609, P1H6, 17E6 and 14D9.F8;

as specific embodiment of the invention, a specific pharmaceutical kit, comprising

(i) a package comprising humanized monoclonal antibody 425 (h425), chimeric monoclonal antibody 225 (c225), or a functionally intact derivative thereof, and

(ii) a package comprising cyclo(Arg-Gly-Asp-DPhe-NMeVal), optionally comprising a chemotherapeutic agent which is selected from any of the compounds of the group: cisplatin, doxorubicin, gemcitabine, docetaxel, paclitaxel, bleomycin;

the use of a pharmaceutical composition or a pharmaceutical kit as defined above, below and in the claims, for the manufacture of a medicament to treat tumors and tumor metastases;

a pharmaceutical treatment or method for treating tumors or tumor metastases in a patient comprising administering to said patient a therapeutically effective amount of an agent or agents having

(i) at least one receptor tyrosine kinase blocking specificity and

(ii) at least one angiogenesis inhibiting specificity, wherein said agent or agents is/are not a cytokine immunoconjugate, optionally together with a cytotoxic, preferably chemotherapeutic agent, and wherein, preferably, said agent (i) is an antibody or a functionally intact derivative thereof, comprising a binding site which binds to an epitope of the ErbB receptor, preferably, ErbB1 (Her1) or Erb2(Her2) receptor, and said agent (ii) is a aᵥβ₃, aᵥβ₅ or an aᵥβ₆ integrin inhibitor or a VEGF receptor blocking agent; and finally

a corresponding method, wherein said antibody directed to the ErbB receptor is selected from the group: humanized monoclonal antibody 425 (h425), chimeric monoclonal antibody 225 (c225) or humanized monoclonal antibody Her 2, and anti-angiogenic agent is cyclo(Arg-Gly-Asp-DPhe-NMeVal), optionally together with a cytotoxic drug selected from the group: cisplatin, doxorubicin, gemcitabine, docetaxel, paclitaxel, bleomycin.

The pharmaceutical treatment using the pharmaceutical compositions and kits according to the invention may be accompanied, concurrently or sequentially, by a radiation therapy.

Principally, four different combinations of pharmaceutical compositions can be distinguished according to the invention:

(i) an agent comprising at least one receptor tyrosine kinase, preferably ErbB receptor blocking activity/specificity combined with an agent comprising at least one anti-angiogenic activity (two-drug combination);

(ii) an agent comprising at least one receptor tyrosine kinase, preferably ErbB receptor blocking activity/specificity combined with an agent comprising at least one anti-angiogenic activity and combined with at least one chemotherapeutic agent (three-drug combination);

(iii) an agent comprising at least one receptor tyrosine kinase, preferably ErbB receptor blocking activity/specificity as well as at least one anti-angiogenic activity combined in one molecule (one-drug combination having two-drug activity);

(iv) an agent comprising at least one receptor tyrosine kinase, preferably ErbB receptor blocking activity/specificity as well as at least one anti-angiogenic activity combined in one molecule, combined with at least one chemotherapeutic agent (two-drug combination having three-drug activity);

The agents can be administered concurrently or sequentially in any of said cases. According to the above-said, the methods of the invention comprise, in principal, the following combinations of administration:

(i) an agent comprising at least one receptor tyrosine kinase, preferably ErbB receptor blocking activity/specificity combined with an agent comprising at least one anti-angiogenic activity (two-drug administration);

(ii) an agent comprising at least one receptor tyrosine kinase, preferably ErbB receptor blocking activity/specificity combined with an agent comprising at least one anti-angiogenic activity (two-drug administration) and radiotherapy;

(iii) an agent comprising at least one receptor tyrosine kinase, preferably ErbB receptor blocking activity/specificity combined with an agent comprising at least one anti-angiogenic activity combined with at least one chemotherapeutic agent (three-drug administration);

(iv) an agent comprising at least one receptor tyrosine kinase, preferably ErbB receptor blocking activity/specificity combined with an agent comprising at least one anti-angiogenic activity combined with at least one chemotherapeutic agent (three-drug administration) and radiotherapy;

(v) an agent comprising at least one receptor tyrosine kinase, preferably ErbB receptor blocking activity/specificity as well as at least one anti-angiogenic activity combined in one molecule (one-drug administration having “two-drug activity”);

(vi) an agent comprising at least one receptor tyrosine kinase, preferably ErbB receptor blocking activity/specificity as well as at least one anti-angiogenic activity combined in one molecule (one-drug administration having “two-drug activity”) and radiotherapy;

(vii) an agent comprising at least one receptor tyrosine kinase, preferably ErbB receptor blocking activity/specificity as well as at least one anti-angiogenic activity combined in one molecule combined with at least one chemotherapeutic agent (two-drug administration having “three-drug activity”);

(viii) an agent comprising at least one receptor tyrosine kinase, preferably ErbB lo receptor blocking activity/specificity as well as at least one anti-angiogenic activity combined in one molecule combined with at least one chemotherapeutic agent (two-drug administration having “three-drug activity”) and radiotherapy.

The pharmaceutical combinations and methods of the present invention provide various benefits. The combinations according to the present invention are useful in treating and preventing tumors, tumor-like and neoplasia disorders. Preferably, the different combined agents of the present invention are administered in combination at a low dose, that is, at a dose lower than has been conventionally used in clinical situations. A benefit of lowering the dose of the compounds, compositions, agents and therapies of the present invention administered to a mammal includes a decrease in the incidence of adverse effects associated with higher dosages. For example, by the lowering the dosage of a chemotherapeutic agent such as methotrexate, doxorubicin, gemcitabine, docetaxel, paclitaxel, bleomycin or cisplatin, a reduction in the frequency and the severity of nausea and vomiting will result when compared to that observed at higher dosages. Similar benefits are contemplated for the compounds, compositions, agents and therapies in combination with the integrin antagonists of the present invention. By lowering the incidence of adverse effects, an improvement in the quality of life of a cancer patient is contemplated. Further benefits of lowering the incidence of adverse effects include an improvement in patient compliance, a reduction in the number of hospitalizations needed for the treatment of adverse effects, and a reduction in the administration of analgesic agents needed to treat pain associated with the adverse effects.

Alternatively, the methods and combination of the present invention can also maximize the therapeutic effect at higher doses.

### DETAILED DESCRIPTION OF THE INVENTION

If not otherwise pointed out the terms and phrases used in this invention have the meanings and definitions as given below. Moreover, these definitions and meanings describe the invention in more detail, preferred embodiments included.

A “receptor” or “receptor molecule” is a soluble or membrane bound/associated protein or glycoprotein comprising one or more domains to which a ligand binds to form a receptor-ligand complex. By binding the ligand, which may be an agonist or an antagonist the receptor is activated or inactivated and may initiate or block pathway signaling.

By “ligand” or “receptor ligand” is meant a natural or synthetic compound which binds a receptor molecule to form a receptor-ligand complex. The term ligand includes agonists, antagonists, and compounds with partial agonist/antagonist action.

An “agonist” or “receptor agonist” is a natural or synthetic compound which binds the receptor to form a receptor-agonist complex by activating said receptor and receptor-agonist complex, respectively, initiating a pathway signaling and further biological processes.

By “antagonist” or “receptor antagonist” is meant a natural or synthetic compound that has a biological effect opposite to that of an agonist. An antagonist binds the receptor and blocks the action of a receptor agonist by competing with the agonist for receptor. An antagonist is defined by its ability to block the actions of an agonist. A receptor antagonist may be also an antibody or an immunotherapeutically effective fragment thereof. Preferred antagonists according to the present invention are cited and discussed below.

An “ErbB receptor” is a receptor protein tyrosine kinase which belongs to the ErbB receptor family and includes EGFR(ErbB1), ErbB2, ErbB3 and ErbB4 receptors and other members of this family to be identified in the future. The ErbB receptor will generally comprise an extracellular domain, which may bind an ErbB ligand; a lipophilic transmembrane domain; a conserved intracellular tyrosine kinase domain; and a carboxyl-terminal signaling domain harboring several tyrosine residues which can be phosphorylated. The ErbB receptor may be a “native sequence” ErbB receptor or an “amino acid sequence variant” thereof. Preferably the ErbB receptor is native sequence human ErbB receptor. ErbB1 refers to the gene encoding the EGFR protein product. Mostly preferred is the EGF receptor (Her 1). The expressions “ErbB1” and “Her 1” are used interchangeably herein and refer to human Her 1 protein. The expressions “ErbB2” and “Her 2” are used interchangeably herein and refer to human Her 2 protein. ErbB1 receptors (EGFR) are preferred according to this invention

“ErbB ligand” is a polypeptide which binds to and/or activates an ErbB receptor. ErbB ligands which bind EGFR include EGF, TGF-a, amphiregulin, betacellulin, HB-EGF and epiregulin.

The term “tyrosine kinase antagonist/inhibitor” refers to natural or synthetic agents that are enabled to inhibit or block tyrosine kinases, receptor tyrosine kinases included, which are of specific interest of this invention. Thus, the term includes “ErbB receptor antagonists/inhibitors”, which are defined below in more detail. With exception of these antagonists, preferably anti-ErbB receptor antibodies additionally suitable tyrosine kinase antagonists of the invention are chemical compounds which have shown efficacy in mono-drug therapy for, e.g., breast and prostate cancer. Suitable indolocarbazole-type tyrosine kinase inhibitors can be obtained using information found in documents such as U.S. Pat. Nos. 5,516,771; 5,654,427; 5,461,146; 5,650,407. U.S. Pat. Nos. 5,475,110; 5,591,855; 5,594,009 and WO 96/11933 disclose pyrrolocarbazole-type tyrosine kinase inhibitors and prostate cancer. Preferably, the dosage of the chemical tyrosine kinase inhibitors as defined above is from 1 pg/kg to 1 g/kg of body weight per day. More preferably, the dosage of tyrosine kinase inhibitors is from 0.01 mg/kg to 100 mg/kg of body weight per day.

The term “ErbB receptor antagonist/inhibitor” refers to a natural or synthetic molecule which binds and blocks or inhibits the ErbB receptor, and is therefore a member of the “(receptor) tyrosine kinase antagonist/inhibitor” family. Thus, by blocking the receptor the antagonist prevents binding of the ErbB ligand (agonist) and activation of the agonist/ligand receptor complex. ErbB antagonists may be directed to Her 1 (or EGFR/Her 1) or Her 2. Preferred antagonists of the invention are directed to the EGF receptor (EGFR, Her 1). The ErbB receptor antagonist may be an antibody or an immunotherapeutically effective fragment thereof or non-immunobiological molecules, such as a peptide, polypeptide protein. Chemical molecules are also included, however, anti-EGFR antibodies and anti-Her 2 antibodies are the preferred antagonists according to the invention. Preferred antibodies of the invention are anti-Her1 and anti-Her2 antibodies, more preferably anti-Her1 antibodies. Preferred anti-Her1 antibodies are MAb 425, preferably humanized MAb 425 (hMAb 425, U.S. Pat. No. 5,558,864; EP 0531 472) and chimeric MAb 225 (cMAb 225, U.S. Pat. No. 4,943,533 and EP 0359 282). Most preferred is monoclonal antibody h425, which has shown in mono-drug therapy high efficacy combined with reduced adverse and side effects. Most preferred anti-Her2 antibody is HERCEPTIN® commercialized by Genentech/Roche.

Efficacious EGF receptor antagonists according to the invention may be also other natural or synthetic chemical compounds. Some examples of preferred molecules of this category include organic compounds, organometallic compounds, salts of organic and organometallic compounds.

Efficacious ErbB receptor antagonists according to the invention may be also small molecules. Small molecules of the invention are not biological molecules as defined above having a molecular weight of approximately not greater than 400. Preferably, they have no protein or peptide structure, and are most preferably synthetically produced chemical compounds. Some examples of preferred small molecules include organic compounds, organometallic compounds, salts of organic and organometallic compounds.

Numerous small molecules have been described as being useful to inhibit EGF receptor and/or Her 2 receptor. Examples are: styryl substituted heteroaryl compounds (U.S. Pat. No. 5,656,655); bis mono and/or bicyclic aryl heteroaryl, carbocyclic, and heterocarbocyclic compounds (U.S. Pat. No. 5,646,153); tricyclic pyrimidine compounds (U.S. Pat. No. 5,679,683); quinazoline derivatives having receptor tyrosine kinase inhibitory activity (U.S. Pat. No. 5,616,582); heteroarylethenediyl or heteroarylethenediylaryl compounds (U.S. Pat. No. 5,196,446); a compound designated as 6-(2,6-dichlorophenyl)-2-(4-(2-diethyl-aminoethoxy) phenylamino)-8-methyl-8H-pyrido(2 3)-5-pyrimidin-7-one (Panek, et al., 1997, J. Pharmacol. Exp. Therap. 283,1433) inhibiting EGFR, PDGFR, and FGFR families of receptors.

An “anti-angiogenic agent” refers to a natural or synthetic compound which blocks, or interferes with to some degree, the development of blood vessels. The anti-angiogenic molecule may, for instance, be a biological molecule that binds to and blocks an angiogenic growth factor or growth factor receptor. The preferred anti-angiogenic molecule herein binds to an receptor, preferably to an integrin receptor or to VEGF receptor. The term includes according to the invention also a prodrug of said angiogenic agent. There are a lot of molecules having different structure and origin which elicit anti-agiogenic properties. Most relevant classes of angiogenesis inhibitong or blocking agents which are suitable in this invention, are, for example:

(i) anti-mitotics such as flurouracil, mytomycin-C, taxol;

(ii) estrogen metabolites such as 2-methoxyestradiol;

(iii) matrix metalloproteinase (MMP) inhibitors, which inhibit zinc metalloproteinases (metalloproteases) (e.g. betimastat, BB16, TIMPs, minocycline, GM6001, or those described in “Inhibition of Matrix Metalloproteinases: Therapeutic Applications” (Golub, Annals of the New York Academy of Science, Vol. 878a; Greenwald, Zucker (Eds.), 1999);

(iv) anti-angiogenic multi-functional agents and factors such as IFNα (U.S. Pat. Nos. 4,530,901; 4,503,035; 5,231,176); angiostatin and plasminogen fragments (e.g. kringle 1-4, kringle 5, kringle 1-3 (O'Reilly, M. S. et al., Cell (Cambridge, Mass.) 79(2): 315-328, 1994; Cao et al., J. Biol Chem. 271: 29461-29467, 1996; Cao et al., J. Biol Chem 272: 22924-22928, 1997); endostatin (O'Reilly, M. S. et al., Cell 88(2), 277, 1997 and WO 97/15666), thrombospondin (TSP-1; Frazier, 1991, Curr Opin Cell Biol 3(5): 792); platelet factor 4 (PF4);

(v) plasminogen activator/urokinase inhibitors;

(vi) urokinase receptor antagonists;

(vii) heparinases;

(viii) fumagillin analogs such as TNP-4701;

(ix) tyrosine kinase inhibitors such as SUI 01 (many of the above and below-mentioned ErbB receptor antagonists (EGFR/Her 2 antagonists) are also tyrosine kinase inhibitors, and may show, therefore anti-EGF receptor blocking activity which results in inhibiting tumor growth, as well as anti-angiogenic activity which results in inhibiting the development of blood vessels and endothelial cells, respectively);

(x) suramin and suramin analogs;

(xi) angiostatic steroids;

(xii) VEGF and bFGF antagonists;

(xiii) VEGF receptor antagonists such as anti-VEGF receptor antibodies (DC-101);

(xiv) flk-1 and fit-1 antagonists;

(xv) cyclooxxygenase-II inhibitors such as COX-II;

(xvi) integrin antagonists and integrin receptor antagonists such as αv antagonists and αv receptor antagonists, for example, anti-αv receptor antibodies and RGD peptides. Integrin (receptor) antagonists are preferred according to this invention.

The term “integrin antagonists/inhibitors” or “integrin receptor antagonists/inhibitors” refers to a natural or synthetic molecule that blocks and inhibit an integrin receptor. In some cases, the term includes antagonists directed to the ligands of said integrin receptors (such as for αᵥβ₃: vitronectin, fibrin, fibrinogen, von Willebrand's factor, thrombospondin, laminin; for αᵥβ₅: vitronectin; for αᵥβ₁: fibronectin and vitronectin; for αᵥβ₆: fibronectin). Antagonists directed to the integrin receptors are preferred according to the invention. Integrin (receptor) antagonists may be natural or synthetic peptides, non-peptides, peptidomimetica, immunoglobulins, such as antibodies or functional fragments thereof, or immunoconjugates (fusion proteins). Preferred integrin inhibitors of the invention are directed to receptor of αᵥ integrins (e.g. αᵥβ₃, αᵥβ₅, αᵥβ₆ and sub-classes). Preferred integrin inhibitors are αᵥ antagonists, and in particular αᵥβ₃ antagonists. Preferred αᵥ antagonists according to the invention are RGD peptides, peptidomimetic (non-peptide) antagonists and anti-integrin receptor antibodies such as antibodies blocking αᵥ receptors. Exemplary, non-immunological αᵥβ₃antagonists are described in the teachings of U.S. Pat. Nos. 5,753,230 and 5,766,591. Preferred antagonists are linear and cyclic RGD-containing peptides. Cyclic peptides are, as a rule, more stable and elicit an enhanced serum half-life. The most preferred integrin antagonist of the invention is, however, cyclo-(Arg-Gly-Asp-DPhe-NMeVal) (EMD 121974, Cilengitide®, Merck KgaA, Germany; EP 0770 622) which is efficacious in blocking the integrin receptors αᵥβ₃, αᵥβ₁, αᵥβ₆, αᵥβ₈, α_{llb}β₃. Suitable peptidyl as well as peptidomimetic (non-peptide) antagonists of the αᵥβ₃/αᵥβ₅/αᵥβ₆ integrin receptor have been described both in the scientific and patent literature. For example, reference is made to Hoekstra and Poulter, 1998, Curr. Med. Chem. 5, 195; WO 95/32710; WO 95/37655; WO 97/01540; WO 97/37655; WO 97/45137; WO 97/41844; WO 98/08840; WO 98/18460; WO 98/18461; WO 98/25892; WO 98/31359; WO 98/30542; WO 99/15506; WO 99/15507; WO 99/31061; WO 00/06169; EP 0853 084; EP 0854 140; EP 0854 145; U.S. Pat. No. 5,780,426; and U.S. Pat. No. 6,048,861. Patents that disclose benzazepine, as well as related benzodiazepine and benzocycloheptene αᵥβ₃ integrin receptor antagonists, which are also suitable for the use in this invention, include WO 96/00574, WO 96/00730, WO 96/06087, WO 96/26190, WO 97/24119, WO 97/24122, WO 97/24124, WO 98/15278, WO 99/05107, WO 99/06049, WO 99/15170, WO 99/15178, WO 97/34865, WO 97/01540, WO 98/30542, WO 99/11626, and WO 99/15508. Other integrin receptor antagonists featuring backbone conformational ring constraints have been described in WO 98/08840; WO 99/30709; WO 99/30713; WO 99/31099; WO 00/09503; U.S. Pat. Nos. 5,919,792; 5,925,655; 5,981,546; and 6,017,926. In U.S. Pat. No. 6,048,861 and WO 00/72801 a series of nonanoic acid derivatives which are potent αᵥβ₃ integrin receptor antagonists were disclosed. Other chemical small molecule integrin antagonists (mostly vitronectin antagonists) are described in WO 00/38665. Other αᵥβ₃ receptor antagonists have been shown to be effective in inhibiting angiogenesis. For example, synthetic receptor antagonists such as (S)-10,11-Dihydro-3-[3-(pyridin-2-ylamino)-1-propyloxy]-5H-dibenzo[a,d]cycloheptene-10-acetic acid (known as SB-265123) have been tested in a variety of mammalian model systems. (Keenan et al., 1998, Bioorg. Med. Chem. Lett. 8(22), 3171; Ward et al., 1999, Drug Metab. Dispos. 27(11),1232). Assays for the identification of integrin antagonists suitable for use as an antagonist are described, e.g. by Smith et al., 1990, J. Biol. Chem. 265, 12267, and in the referenced patent literature. Anti-integrin receptor antibodies are also well known. Suitable anti-integrin (e.g. αᵥβ₃, αᵥβ₅, αᵥβ₆) monoclonal antibodies can be modified to encompasses antigen binding fragments thereof, including F(ab)₂, Fab, and engineered Fv or single-chain antibody. One suitable and preferably used monoclonal antibody directed against integrin receptor Cav3 is identified as LM609 (Brooks et al., 1994, Cell 79, 1157; ATCC HS 9537). A potent specific anti-αᵥβ₅ antibody, P1F6, is disclosed in WO 97/45447, which is also preferred according to this invention. A further suitable αᵥβ₆ selective antibody is MAb 14D9.F8 (WO 99/37683, DSM ACC2331, Merck KGaA, Germany) as well as MAb 17.E6 (EP 0719 859, DSM ACC2160, Merck KGaA) which is selectively directed to the αᵥ-chain of integrin receptors. Another suitable anti-integrin antibody is the commercialized Vitraxin ®.

The term “antibody” or “immunoglobulin” herein is used in the broadest sense and specifically covers intact monoclonal antibodies, polyclonal antibodies, multispecific antibodies (e.g. bispecific antibodies) formed from at least two intact antibodies, and antibody fragments, so long as they exhibit the desired biological activity. The term generally includes heteroantibodies which are composed of two or more antibodies or fragments thereof of different binding specificity which are linked together. Depending on the amino acid sequence of their constant regions, intact antibodies can be assigned to different “antibody (immunoglobulin) classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain conscant domains that correspond to the different classes of antibodies are called α, δ, ε, γ and μ respectively. Preferred major class for antibodies according to the invention is IgG, in more-detail IgG1 and IgG2. Antibodies are usually glycoproteins having a molecular weight of about 150,000, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (VH) followed by a number of constant domains. The variable regions comprise hypervariable regions or “CDR” regions, which contain the antigen binding site and are responsible for the specificity of the antibody, and the “FR” regions, which are important with respect to the affinity/avidity of the antibody. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; and/or those residues from a “hypervariable loop” (e.g. residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). The “FR” residues (frame work region) are those variable domain residues other than the hypervariable region residues as herein defined. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. Methods for making monoclonal antibodies include the hybridoma method described by Kohler and Milstein (1975, Nature 256, 495) and in “Monoclonal Antibody Technology, The Production and Characterization of Rodent and Human Hybridomas” (1985, Burdon et al., Eds, Laboratory Techniques in Biochemistry and Molecular Biology, Volume 13, Elsevier Science Publishers, Amsterdam), or may be made by well known recombinant DNA methods (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:58, 1-597(1991), for example.

The term “chimeric antibody” means antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (e.g.: U.S. Pat. No. 4,816,567; Morrison et al., Proc. Nat. Acad. Sci. USA, 81:6851-6855 (1984)). Methods for making chimeric and humanized antibodies are also known in the art. For example, methods for making chimeric antibodies include those described in patents by Boss (Celltech) and by Cabilly (Genentech) (U.S. Pat. No. 4,816,397; U.S. Pat. No. 4,816,567).

“Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

“Antibody fragments” comprise a portion of an intact antibody, preferably comprising the antigen-binding or variable region thereof. Examples of antibody fragments include Fab, Fab′, F(ab′)2, Fv and Fc fragments, diabodies, linear antibodies, single-chain antibody molecules; and multispecific antibodies formed from antibody fragment(s). An “intact” antibody is one which comprises an antigen-binding variable region as well as a light chain constant domain (CL) and heavy chain constant domains, CH1, CH2 and CH3. Preferably, the intact antibody has one or more effector functions. Papain digestion of antibodies produces two identical antigen-binding fragments, called “Fab” fragments, each comprising a single antigen-binding site and a CL and a CH1 region, and a residual. “Fc” fragment, whose name reflects its ability to crystallize readily. The “Fc” region of the antibodies comprises, as a rule, a CH2, CH3 and the hinge region of an IgG1 or IgG2 antibody major class. The hinge region is a group of about 15 amino acid residues which combine the CH1 region with the CH2-CH3 region. Pepsin treatment yields an “F(ab′)2” fragment that has two antigen-binding sites and is still capable of cross-linking antigen. “Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions (CDRs) of each variable domain interact to define an antigen-binding site on the surface-of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site. The Fab fragment also contains the constant domain of the light chain and the first constant domain (CH1) of the heavy chain. “Fab′” fragments differ from Fab fragments by the addition of a few residues at the carboxy terminus of the heavy chain CH1 domain including one or more cysteines from the antibody hinge region. F(ab′)2 antibody fragments originally were produced as pairs of Fab′ fragments which have hinge cysteines between them. Other chemical couplings of antibody fragments are also known (see e.g. Hermanson, Bioconjugate Techniques, Academic Press, 1996; U.S. Pat. No. 4,342,566). “Single-chain Fv” or “scFv” antibody fragments comprise the V, and V, domains of antibody, wherein these domains are present in a Single polypeptide chain. Preferably, the Fv polypeptide further comprises a polypeptide linker between the VH and VL domains which enables the scFv to form the desired structure for antigen binding. Single-chain FV antibodies are known, for example, from Plückthun (The Pharmacology of Monoclonal Antibodies, Vol. 113, Rosenburg and Moore eds., Springer-Verlag, New York, pp. 269-315 (1994)), W093/16185; U.S. Pat. No. 5,571,894; U.S. Pat. No. 5,587,458; Huston et al. (1988, Proc.Natl. Acad. Sci. 85, 5879) or Skerra and Plueckthun (1988, Science 240, 1038).

“Bispecific antibodies” are single, divalent antibodies (or immunotherapeutically effective fragments thereof) which have two differently specific antigen binding sites. For example the first antigen binding site is directed to an angiogenesis receptor (e.g. integrin or VEGF receptor), whereas the second antigen binding site is directed to an ErbB receptor (e.g. EGFR or Her 2). Bispecific antibodies can be produced by chemical techniques (see e.g., Kranz et al. (1981) Proc. Natl. Acad. Sci. USA 78, 5807), by “polydoma” techniques (See U.S. Pat. No. 4,474,893) or by recombinant DNA techniques, which all are known per se. Further methods are described in WO 91/00360, WO 92/05793 and WO 96/04305. Bispecific antibodies can also be prepared from single chain antibodies (see e.g., Huston et al. (1988) Proc. Natl. Acad. Sci. 85, 5879; Skerra and Plueckthun (1988) Science 240, 1038). These are analogues of antibody variable regions produced as a single polypeptide chain. To form the bispecific binding agent, the single chain antibodies may be coupled together chemically or by genetic engineering methods known in the art. It is also possible to produce bispecific antibodies according to this invention by using leucine zipper sequences. The sequences employed are derived from the leucine zipper regions of the transcription factors Fos and Jun (Landschulz et al., 1988, Science 240, 1759; for review, see Maniatis and Abel, 1989, Nature 341, 24). Leucine zippers are specific amino acid sequences about 20-40 residues long with leucine typically occurring at every seventh residue. Such zipper sequences form amphipathic α-helices, with the leucine residues lined up on the hydrophobic side for dimer formation. Peptides corresponding to the leucine zippers of the Fos and Jun proteins form heterodimers preferentially (O'Shea et al., 1989, Science 245, 646). Zipper containing bispecific antibodies and methods for making them are also disclosed in WO 92/10209 and WO 93/11162. A bispecific antibody according the invention may be an antibody, directed to VEGF receptor and αVβ3 receptor as discussed above with respect to the antibodies having single specificity.

“Heteroantibodies” are two or more antibodies or antibody-binding fragments which are linked together, each of them having a different binding specificity. Heteroantibodies can be prepared by conjugating together two or more antibodies or antibody fragments. Preferred heteroantibodies are comprised of cross-linked Fab/Fab′ fragments. A variety of coupling or crosslinking agents can be used to conjugate the antibodies. Examples are protein A, carboiimide, N-succinimidyl-S-acetyl-thioacetate (SATA) and N-succinimidyl-3-(2-pyridyldithio) propionate (SPDP) (see e.g., Karpovsky et al. (1984) J. EXP. Med. 160, 1686; Liu et a. (1985) Proc. Natl. Acad. Sci. USA 82, 8648). Other methods include those described by Paulus, Behring Inst. Mitt., No. 78, 118 (1985); Brennan et a. (1985) Science 30 m:81 or Glennie et al. (1987) J. Immunol. 139, 2367. Another method uses o-phenylenedimaleimide (oPDM) for coupling three Fab′ fragments (WO 91/03493). Multispecific antibodies are in context of this invention also suitable and can be prepared, for example according to the teaching of WO 94/13804 and WO 98/50431.

The term “fusion protein” refers to a natural or synthetic molecule consisting of one ore more proteins or peptides or fragments thereof having different specificity which are fused together optionally by a linker molecule. As specific embodiment the term includes fudsion constructs, wherein at least one protein or peptide is a immunoglubulin or antibody, respectively or parts thereof (“immunoconjugates”).

The term “immunoconjugate” refers to an antibody or immunoglobulin respectively, or a immunologically effective fragment thereof, which is fused by covalent linkage to a non-immunologically effective molecule. Preferably this fusion partner is a peptide or a protein, which may be glycosylated. Said non-antibody molecule can be linked to the C-terminal of the constant heavy chains of the antibody or to the N-terminals of the variable light and/or heavy chains. The fusion partners can be linked via a linker molecule, which is, as a rule, a 3-15 amino acid residues containing peptide. Immunoconjugates according to the invention consist of an immunoglobulin or immunotherapeutically effective fragment thereof, directed to a receptor tyrosine kinase, preferably an ErbB (ErbB1/ErbB2) receptor and an integrin antagonistic peptide, or an angiogenic receptor, preferably an integrin or VEGF receptor and TNFα or a fusion protein consisting essentially of TNFα and IFNγ or another suitable cytokine, which is linked with its N-terminal to the C-terminal of said immunoglobulin, preferably the Fc portion thereof. The term includes also corresponding fusion constructs comprising bi- or multi-specific immunoglobulins (antibodies) or fragments thereof.

The term “functionally intact derivative” means according to the understanding of this invention a fragment or portion, modification, variant, homologue or a de-immunized form (a modification, wherein epitopes, which are responsible for immune responses, are removed) of a compound, peptide, protein, antibody (immunoglobulin), immunconjugate, etc., that has principally the same biological and/or therapeutic function as compared with the original compound, peptide, protein, antibody (immunoglobulin), immunconjugate, etc. However, the term includes also such derivatives, which elicit a reduced or enhanced efficacy.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone ,(TSH), and luteinizing hormone (LH); hepatic growth factor; fibroblast growth factor; prolactin; placental lactogen; mouse gonadotropin-associated peptide; inhibin; activin; vascular endothelial growth factor (VEGF); integrin; thrombopoietin (TPO); nerve growth factors such as NGFβ; platelet-growth factor; transforming growth factors (TGFs) such as TGFα and TGFβ; erythropoietin (EPO); interferons such as IFNα, IFNβ, and IFNγ; colony stimulating factors such as M-CSF, GM-CSF and G-CSF; interleukins such as IL-1, IL-1a, IL-2, IL-3, IL-4, IL-5, IL-6, IL-7, IL-8, IL-9, IL-10, IL-11,IL12; and TNFα or TNFβ. Preferred cytokines according to the invention are interferons and TNFa.

The term “cytotoxic agent” as used herein refers to a substance that inhibits or prevents the function of cells and/or causes destruction of cells. The term is intended to include radioactive isotopes, chemotherapeutic agents, and toxins such as enzymatically active toxins of bacterial, fungal, plant or animal origin, or fragments thereof. The term may include also members of the cytokine family, preferably IFNγ as well as anti-neoplastic agents having also cytotoxic activity.

The term “chemotherapeutic agent” or “anti-neoplastic agent” is regarded according to the understanding of this invention as a member of the class of “cytotoxic agents”, as specified above, and includes chemical agents that exert anti-neoplastic effects, i.e., prevent the development, maturation, or spread of neoplastic cells, directly on the tumor cell, e.g., by cytostatic or cytotoxic effects, and not indirectly through mechanisms such as biological response modification. Suitable chemotherapeutic agents according to the invention are preferably natural or synthetic chemical compounds, but biological molecules, such as proteins, polypeptides etc. are not expressively excluded. There are large numbers of anti-neoplastic agents available in commercial use, in clinical evaluation and in pre-clinical development, which could be included in the present invention for treatment of tumors/neoplasia by combination therapy with TNFα and the anti-angiogenic agents as cited above, optionally with other agents such as EGF receptor antagonists. It should be pointed out that the chemotherapeutic agents can be administered optionally together with above-said drug combination. Examples of chemotherapeutic or agents include alkylating agents, for example, nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic antibiotics and camptothecin derivatives. Preferred chemotherapeutic agents or chemotherapy include amifostine (ethyol), cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa,. uracil mustard, vinorelbine, chlorambucil and combinations thereof.

Most preferred chemotherapeutic agents according to the invention are cisplatin, gemcitabine, doxorubicin, paclitaxel (taxol) and bleomycin.

The terms “cancer” and “tumor” refer to or describe the physiological condition in mammals that is typically characterized by unregulated cell growth. By means of the pharmaceutical compositions according of the present invention tumors can be treated such as tumors of the breast, heart, lung, small intestine, colon, spleen, kidney, bladder, head and neck, ovary, prostate, brain, pancreas, skin, bone, bone marrow, blood, thymus, uterus, testicles, cervix, and liver. More specifically the tumor is selected from the group consisting of adenoma, angio-sarcoma, astrocytoma, epithelial carcinoma, germinoma, glioblastoma, glioma, hamartoma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and teratoma. In detail, the tumor is selected from the group consisting of acral lentiginous melanoma, actinic keratoses, adenocarcinoma, adenoid cycstic carcinoma, adenomas, adenosarcoma, adenosquamous carcinoma, astrocytic tumors, bartholin gland carcinoma, basal cell carcinoma, bronchial gland carcinomas, capillary, carcinoids, carcinoma, carcinosarcoma, cavernous, cholangio-carcinoma, chondosarcoma, choriod plexus papilloma/carcinoma, clear cell carcinoma, cystadenoma, endodermal sinus tumor, endometrial hyperplasia, endometrial stromal sarcoma, endometrioid adenocarcinoma, ependymal, epitheloid, Ewing's sarcoma, fibrolamellar, focal nodular hyperplasia, gastrinoma, germ cell tumors, glioblastoma, glucagonoma, hemangiblastomas, hemangioendothelioma, hemangiomas, hepatic adenoma, hepatic adenomatosis, hepatocellular carcinoma, insulinoma, intaepithelial neoplasia, interepithelial squamous cell neoplasia, invasive squamous cell carcinoma, large cell carcinoma, leiomyosarcoma, lentigo maligna melanomas, malignant melanoma, malignant mesothelial tumors, medulloblastoma, medulloepithelioma, melanoma, meningeal, mesothelial, metastatic carcinoma, mucoepidermoid carcinoma, neuroblastoma, neuroepithelial adenocarcinoma nodular melanoma, oat cell carcinoma, oligodendroglial, osteosarcoma, pancreatic polypeptide, papillary serous adeno-carcinoma, pineal cell, pituitary tumors, plasmacytoma, pseudo-sarcoma, pulmonary blastoma, renal cell carcinoma, retinoblastoma, rhabdomyo-sarcoma, sarcoma, serous carcinoma, small cell carcinoma, soft tissue carcinomas, somatostatin-secreting tumor, squamous carcinoma, squamous cell carcinoma, submesothelial, superficial spreading melanoma, undifferentiated carcinoma, uveal melanoma, verrucous carcinoma, vipoma, well differentiated carcinoma, and Wilm's tumor.

The “pharmaceutical compositions” of the invention can comprise agents that reduce or avoid side effects associated with the combination therapy of the present invention (“adjunctive therapy”), including, but not limited to, those agents, for example, that reduce the toxic effect of anticancer drugs, e.g., bone resorption inhibitors, cardioprotective agents. Said adjunctive agents prevent or reduce the incidence of nausea and vomiting associated with chemotherapy, radiotherapy or operation, or reduce the incidence of infection associated with the administration of myelosuppressive anticancer drugs. Adjunctive agents are well known in the art. The immunotherapeutic agents according to the invention can additionally administered with adjuvants like BCG and immune system stimulators. Furthermore, the compositions may include immunotherapeutic agents or chemotherapeutic agents which contain cytotoxic effective radio labeled isotopes, or other cytotoxic agents, such as a cytotoxic peptides (e.g. cytokines) or cytotoxic drugs and the like.

The term “pharmaceutical kit” for treating tumors or tumor metastases refers to a package and, as a rule, instructions for using the reagents in methods to treat tumors and tumor metastases. A reagent in a kit of this invention is typically formulated as a therapeutic composition as described herein, and therefore can be in any of a variety of forms suitable for distribution in a kit. Such forms can include a liquid, powder, tablet, suspension and the like formulation for providing the antagonist and/or the fusion protein of the present invention. The reagents may be provided in separate containers suitable for administration separately according to the present methods, or alternatively may be provided combined in a composition in a single container in the package. The package may contain an amount sufficient for one or more dosages of reagents according to the treatment methods described herein. A kit of this invention also contains “instruction for use” of the materials contained in the package.

The term “pharmaceutical treatment” refers to therapeutic methods of the present invention for treating tumor cells in tumors and tumor metastases are based on the combined use of angiogenesis inhibiting (anti-angiogenesis) therapy and anti-tumor immunotherapy by using receptor tyrosine kinase blocking agents, preferably ErbB antagonists, above all anti-ErbB1(EGFR, Her1)/anti-ErbB2 (Her2) antibodies. More than one type of angiogenesis inhibiting agent can be used in combination with more than one type of preferably anti-ErbB receptor inhibiting agent. The combined use can occur simultaneously, sequentially, or with the intervention of a period of time between the treatments. Any of the specific therapeutics may be administered more than once during a course of treatment. The method can result in a synergistic potentiation of the tumor cell proliferation inhibition effect of each individual therapeutic, yielding more effective treatment than found by administering an individual component alone. Thus, in one aspect, the method of the invention encompasses administering to a patient, in combination, an amount of an anti-angiogenic agent and an anti-ErbB receptor (Her1/Her2) agent, that may not result in effective angiogenesis inhibition, or anti-tumor cell activity if given in that amount alone. The method of the invention comprises a variety of modalities for practicing the invention in terms of the steps. For example, the agents according to the invention can be administered simultaneously, sequentially, or separately. Furthermore, the receptor tyrosine kinase blocking agent and the anti-angiogenic agent can be separately administered within a time interval of about 3 weeks between administrations, i.e., from substantially immediately after the first active agent is administered to up to about 3 weeks after the first agent is administered. The method can be practiced following a surgical procedure. Alternatively, the surgical procedure can be practiced during the interval between administration of the first active agent and the second active agent. Exemplary of this method is the combination of the present method with surgical tumor removal. Treatment according to the method will typically comprise administration of the therapeutic compositions in one or more cycles of administration. For example, where a simultaneous administration is practiced, a therapeutic composition comprising both agents is administered over a time period of from about 2 days to about 3 weeks in a single cycle. Thereafter, the treatment cycle can be repeated as needed according to the judgment of the practicing physician. Similarly, where a sequential application is contemplated, the administration time for each individual therapeutic will be adjusted to typically cover the same time period. The interval between cycles can vary from about zero to 2 months. The monoclonal antibodies, polypeptides or organic mimetics/chemotherapeutics of this invention can be administered parenterally by injection or by gradual infusion over time. Although the tissue to be treated can typically be accessed in the body by systemic administration and therefore most often treated by intravenous administration of therapeutic compositions, other tissues and delivery means are contemplated where there is a likelihood that the tissue targeted contains the target molecule. Thus, monoclonal antibodies, polypeptidcs or organic agents of this invention can be administered intraocularly, intravenously, intraperitoneally, intramuscularly, subcutaneously, intracavity, transdermally, by orthotopic injection and infusion, and can also be delivered by peristaltic means. The therapeutic compositions containing, for example, an integrin antagonist of this invention are conventionally administered intravenously, as by injection of a unit dose, for example. Therapeutic compositions of the present invention contain a physiologically tolerable carrier together with the relevant agent as described herein, dissolved or dispersed therein as an active ingredient.

As used herein. the terms “pharmaceutically acceptable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. The preparation of a pharmacological composition that contains active ingredients dissolved or dispersed therein is well understood in the art and need not be limited based on formulation. Typically, such compositions are prepared as injectables either as liquid solutions or suspensions, however, solid forms suitable for solution, or suspensions, in liquid prior to use can also be prepared. The preparation can also be emulsified. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents., pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as. for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Particularly preferred is the HCI salt when used in the preparation of cyclic polypeptide αv antagonists. Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin. vegetable oils such as cottonseed oil, and water-oil emulsions.

Typically, a therapeutically effective amount of an immunotherapeutic agent in the form of a, for example, anti-ErbB receptor antibody or antibody fragment or antibody conjugate or an anti-angiogenic receptor antibody, fragment or-conjugate is an amount such that when administered in physiologically tolerable composition is sufficient to achieve a plasma concentration of from about 0.01 microgram (μg) per milliliter (ml) to about 100 μg/ml, preferably from about 1 μg/ml to about 5 μg/ml and usually about 5 μg/ml. Stated differently. the dosage can vary from about 0.1 mg/kg to about 300 mg/kg, preferably from about 0.2 mg/kg to about 200 mg/kg, most preferably from about 0.5 mg/kg to about 20 mg/kg, in one or more dose administrations daily for one or several days. Where the immunotherapeutic agent is in the form of a fragment of a monoclonal antibody or a conjugate, the amount can readily be adjusted based on the mass of the fragment/conjugate relative to the mass of the whole antibody. A preferred plasma concentration in molarity is from about 2 micromolar (μM) to about 5 millimolar (mM) and preferably, about 100 μM to 1 mM antibody antagonist. A therapeutically effective amount of an agent according of this invention which is a non-immunotherapeutic peptide or a protein polypeptide (e.g. IFN-alpha), or other similarly-sized small molecule, is typically an amount of polypeptide such that when administered in a physiologically tolerable composition is sufficient to achieve a plasma concentration of from about 0.1 microgram (μg) per milliliter (ml) to about 200 μg/ml, preferably from about 1 μg/ml to about 150 μg/ml. Based on a polypeptide having a mass of about 500 grams per mole, the preferred plasma concentration in molarity is from about 2 micromolar (μM) to about 5 millimolar (mM) and preferably about 100 μM to 1 mM polypeptide antagonist. The typical dosage of an active agent, which is a preferably a chemical antagonist or a (chemical) chemotherapeutic agent according to the invention (neither an immunotherapeutic agent nor a non-immunotherapeutic peptide/protein) is 10 mg to 1000 mg, preferably about 20 to 200 mg, and more preferably 50 to 100 mg per kilogram body weight per day.

The term “therapeutically effective” or “therapeutically effective amount” refers to an amount of a drug effective to treat a disease or disorder in a mammal. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

### EXAMPLE

The Following is a Short Clinical Trial Report:

A patient, 45 years old, was originally suffering from progressive squamous cell carcinoma of the superior maxilla.

| | |
|---|---|
| EMD 72000: | Monoclonal human antibody 425 (h425), |
| | Merck KgaA, Germany |
| EMD 121974: | Cyclo-(Arg-Gly-Asp-DPhe-NMeVal), |
| | Cilengitide ®, Merck KgaA, Germany; |
| Chemotherapeutics: | various, (gemcitabine cisplatin, etc.) |

Case History and Clinical Findings/status at the Start of the Compassionate use Treatment:

In July 1997 the patient first presented at the Virchow Klinikum, Germany. A biopsy of the suspected large tumor in the upper maxilla was performed. Histology revealed a squamous cell carcinoma classified as T4 N0 M0. On Aug. 5, 1997 partial resection of the superior maxilla and resection of regional lymph nodes was done. Histology revealed that no clean margin had been achieved, so an additional resection was performed during the same hospital stay. Due to the unfavourable histologic classification the patient received a post-operative radiation therapy up to 50.4 Gray from September to October 1997.

In July 1998 progression of disease was suspected which led to hospitalization. Histology now showed an adenosquamous carcinoma. After consultation of radiotherapists another radiotherapy was recommended which started in August 1998. The patient was treated simultaneously with gemcitabine (100 mg) as a radiosensitizer. The 6 week-therapy led to complete clinical remission. Following the combined radio-chemotherapy the patient received a therapy with 1000 mg gemcitabine (5 circles of 16 administrations).

In March 1999 again progression of the carcinoma occurred which led to additional radiation therapy and palliative resection of the tumor. In August 1999 again tumor progression and chemotherapy with cisplatin (75 mg/m²) and docetaxel (75 mg/m²) was started. After three administrations the therapy was stopped due to lack of effect on tumor growth.

Diffuse bleedings out of the large tumor mass required frequent transfusions of erythrocyte concentrates.

Course of Compassionate use Treatment with Anti-angiogenic Agent/chemotherapeutic Agents:

Under treatment with EMD 121974 (600 mg/m²) and gemcitabine (Gemzar) (1000 mg/m²) in November 1999 a regression of the tumor was diagnosed. Since mid of January 2000 the patient had been able to hear again on his right ear and he had been able to open his mouth 30% more than in December 1999. The surface of the tumor showed signs of granulation and wound healing. Bleeding stopped and there was no need for further transfusions.

The patient was treated with EMD 121974 and Gemzar from Nov. 17, 1999 until Mar. 30, 2000. From Apr. 6, 2000 until Apr. 28, 2000 EMD 121974, Gemzar and a chemotherapy with 5-FU, cisplatin and rescuvolin was given to the patient because a progression of the tumor was detected. Chemotherapy-treatment was stopped because of haematotoxicity and Cilengitide treatment was continued alone. From April to June 2000 the patient received 600 mg/m² EMD 121974 twice a week only resulting in stable disease.

The patient's condition worsened after several weeks and the patient was treated with an increased dose of 1200 mg/m² EMD 121974 twice weekly.

Treatment with h425 +Cilenqitide +Chemotherapeutics:

EMD 72000 was first given in November 2000 in a dosage of 200 mg (infusion over half an hour) after premedication with dexamethasone/dimetindenmaleate (Fenistil) and ranitidin (Zantic). One week later the patient received additionally gemcitabine (1000 mg/m²). The weekly treatment schedule was: Monday:1200mg/m² Cilengitide (one hour infusion),Thursday 200 mg EMD 72 000 (half an hour infusion) followed by 1000 mg/m² gemcitabine (one hour infusion), Friday 1200 mg/m² Cilengitide (one hour infusion). Under this treatment a crater-like disintegration of the tumor mass was observed. The tumor masses were surgically removed at several occasions. The effect of the combined treatment was considered exceptionally impressive by the treating physicians. No therapy related adverse drug reactions in relation to EMD 121974 and EMD 72000 were observed. Up to now the patient's condition remained improved.

1. A pharmaceutical composition comprising an agent or agents having
(i) at least one receptor tyrosine kinase blocking specificity and
(ii) at least one angiogenesis inhibiting specificity, wherein said agent or agents is/are not a cytokine immunoconjugate, optionally together with a pharmaceutically acceptable carrier, diluent or recipient.

2. A pharmaceutical composition of claim 1, further comprising a cytotoxic agent.

3. A pharmaceutical composition of claim 1 or 2, comprising
(i) at least one agent having a receptor tyrosine kinase blocking specificity, and
(ii) at least one agent having an angiogenesis inhibiting specificity.

4. A pharmaceutical composition of claim 1 or 2, comprising an agent having a receptor tyrosine kinase blocking specificity as well as an angiogenesis inhibiting specificity.

5. A pharmaceutical composition of claim 3, wherein said agent (i) has a ErbB receptor blocking specificity.

6. A pharmaceutical composition of claim 5, wherein the ErbB receptor specificity of said agent is related to the EGF receptor (ErbB1/Her1) or the ErbB2/Her2 receptor.

7. A pharmaceutical composition according to claim 6, wherein said agent is an antibody or a functionally intact derivative thereof, comprising a binding site which binds to an epitope of the ErbB1 (Her1) or Erb2(Her2) receptor.

8. A pharmaceutical composition according to claim 7, wherein said antibody or functionally intact derivative thereof is selected from the group:
humanized monoclonal antibody 425 (h425)
chimeric monoclonal antibody 225 (c225)
humanized monoclonal antibody Her 2.

9. A pharmaceutical composition according to any of the claims 1-8, wherein said angiogenesis inhibiting agent is an αᵥβ₃, αᵥβ₅ or an αᵥβ₆ integrin inhibitor.

10. A pharmaceutical composition according to claim 9, wherein said integrin inhibitor is an RGD-containing linear or cyclic peptide.

11. A pharmaceutical composition according to claim 10, wherein said peptide is cyclo(Arg-Gly-Asp-DPhe-NMeVal).

12. A pharmaceutical composition according to claims 7 and 9, wherein said antibody or functionally intact derivative thereof is humanized monoclonal antibody 425 (h425) or chimeric monoclonal antibody 225 (c225) and said integrin inhibitor is cyclo(Arg-Gly-Asp-DPhe-NMeVal).

13. A pharmaceutical composition according to claim 12, further comprising a chemotherapeutic agent which is selected from any of the compounds of the group: cisplatin, doxorubicin, gemcitabine, docetaxel, paclitaxel, bleomycin.

14. A pharmaceutical composition according to claim 9, wherein said integrin inhibitor is an antibody or a functionally intact derivative thereof, comprising a binding site which binds to an epitope of an integrin receptor.

15. A pharmaceutical composition according to claim 14, wherein said antibody is LM609 or P1F6.

16. A pharmaceutical composition according to claim 4, wherein said agent is a bispecific antibody or a heteroantibody molecule comprising a first binding site that binds to an epitope of a receptor tyrosine kinase and a second binding site that binds to an epitope of an angiogenesis receptor.

17. A pharmaceutical composition according to claim 16, wherein said bispecific antibody or heteroantibody molecule comprises a first binding site that binds to an epitope of an ErbB receptor and a second binding site that binds to an epitope of an integrin receptor.

18. A pharmaceutical composition according to claim 17, wherein said binding sites, which bind to an epitope of an ErbB receptor, are selected from monoclonal antibodies h425, c225 or Her 2, and said binding sites, which bind to an epitope of an integrin receptor, are selected from the monoclonal antibodies LM609 or P1F6.

19. A pharmaceutical composition according to claim 4, wherein said agent is an immunoconjugate consisting of an antibody or antibody fragment, bearing one of said specificities, and a non-immunological molecule, fused to the antibody or antibody fragment bearing the other specificity.

20. A pharmaceutical composition according to claim 19, wherein the antibody portion or fragment thereof comprises a binding site that binds to an epitope of an ErbB receptor, and the fused non-immunological molecule comprises a binding site that binds to an epitope of an integrin receptor.

21. A pharmaceutical composition according to claim 20, wherein said antibody portion which binds to an epitope of an ErbB receptor is selected from monoclonal antibodies h425, c225 or Her 2, and said non-imunological portion which binds to an epitope of an integrin receptor is cyclo(Arg-Gly-Asp-DPhe-NMeVal).

22. A pharmaceutical kit comprising
(i) a package comprising at least one ErbB receptor blocking agent, and
(ii) a package comprising at least one angiogenesis inhibiting agent.

23. A pharmaceutical kit of claim 22, further comprising a package comprising a cytotoxic agent.

24. A pharmaceutical kit comprising
(i) a package comprising at least one ErbB receptor blocking agent and at least one angiogenesis inhibiting agent, and
(ii) a package comprising a cytotoxic agent.

25. The pharmaceutical kit of any of the claims 22-24, wherein said ErbB receptor blocking agent is an antibody or a functionally intact derivative thereof, having a binding site that binds to an epitope of said receptor.

26. A pharmaceutical kit of claim 25, wherein said antibody or functionally intact derivative thereof is selected from the group: humanized monoclonal antibody 425 (h425), chimeric monoclonal antibody 225 (c225) or humanized monoclonal antibody Her 2.

27. A pharmaceutical kit of any of the claims 22-26, wherein said angiogenesis inhibiting agent is an αᵥβ₃, αᵥβ₅ or an αᵥβ₆ integrin inhibitor.

28. A pharmaceutical kit of claim 27, wherein said integrin inhibitor is an RGD-containing linear or cyclic peptide.

29. A pharmaceutical kit of claim 28, wherein said peptide is cyclo(Arg-Gly-Asp-DPhe-NMeVal).

30. A pharmaceutical kit of any of the claims 22-26, wherein said angiogenesis inhibiting agent is an antibody or a functionally intact derivative thereof.

31. A pharmaceutical kit of claim 30, wherein said antibody is LM609 or P1F6.

32. A pharmaceutical kit of claim 22, comprising
(i) a package comprising humanized monoclonal antibody 425 (h425), chimeric monoclonal antibody 225 (c225), or a functionally intact derivative thereof, and
(ii) a package comprising cyclo(Arg-Gly-Asp-DPhe-NMeVal).

33. A pharmaceutical kit of claim 32, further comprising a chemotherapeutic agent which is selected from any of the compounds of the group: cisplatin, doxorubicin, gemcitabine, docetaxel, paclitaxel, bleomycin

34. Use of a pharmaceutical composition or a pharmaceutical kit as defined in any of the claims 1-33, for the manufacture of a medicament to treat tumors and tumor metastases.

35. A method for treating tumors or tumor metastases in a patient comprising administering to said patient a therapeutically effective amount of an agent or agents having
(i) at least one receptor tyrosine kinase blocking specificity and
(ii) at least one angiogenesis inhibiting specificity,
wherein said agent or agents is/are not a cytokine immunoconjugate.

36. A method of claim 35, wherein additionally a cytotoxic agent is administered to the patient.

37. A method of claim 35 or 36, wherein said agent or agents have a receptor tyrosine kinase blocking specificity which is related to the ErbB receptor family.

38. A method of claim 37, wherein the ErbB receptor specificity of said agent is related to the EGF receptor (ErbB1/Her1) or the ErbB2/Her2 receptor.

39. A method of claim 38, wherein said agent is an antibody or a functionally intact derivative thereof, comprising a binding site which binds to an epitope of the ErbB1(Her1) or Erb2(Her2) receptor.

40. A method of claim 39, wherein said antibody or derivative thereof is selected from the group: humanized monoclonal antibody 425 (h425), chimeric monoclonal antibody 225 (c225) or humanized monoclonal antibody Her 2.

41. A method of any of the claims 35-40, wherein said angiogenesis inhibiting agent is an αᵥβ₃, αᵥβ₅ or an αᵥβ₆ integrin inhibitor or a VEGF receptor blocking agent.

42. A method of claim 41, comprising administering to the patient a therapeutically effective amount of (i) humanized monoclonal antibody 425 (h425) or chimeric monoclonal antibody 225 (c225), (ii) cyclo(Arg-Gly-Asp-DPhe-NMeVal), and optionally (iii) cisplatin, doxorubicin, gemcitabine, docetaxel, paclitaxel, bleomycin.
